(19) United States
(12) Patent Application Publication
(10) Pub. No.: US 2018/0021721 A1
(43) Pub. Date: Jan. 25, 2018

(54) MAGNETIC DECOMPOSITION DEVICE, AND MAGNETIC DECOMPOSITION METHOD

(71) Applicant: SHINEI METALTEC CO., LTD., Saga (JP)

(72) Inventors: Kazuyuki TABARU, Saga (JP); Kyongil CHIN, Saga (JP)

(21) Appl. No.: 15/549,105

(22) PCT Filed: Feb. 4, 2016
(86) PCT No.: PCT/JP2016/053362

§ 371 Date: Aug. 4, 2017

(30) Foreign Application Priority Data
Feb. 6, 2015 (JP) 2015-022411

(51) Int. Cl. B01D 53/34; B01J 19/08; B01D 51/00; A61L 9/16

(52) U.S. Cl. CPC B01D 53/34; A61L 9/16; B01J 19/087; B01D 51/00; F23G 5/027

(57) ABSTRACT

[Object] To perform a magnetic decomposition treatment by effectively utilizing smoke emissions.

[Solution Means] When an opening 112 is opened and hot air is blown by a hot air gun 40, the waste is thermally decomposed inside the thermal decomposition treatment chamber 110. When smoke emissions generated by the thermal decomposition pass through a shower 210 and a swashplate filter 220 of the filter section 200, a temperature thereof is lowered and tar is removed, and then the smoke emissions pass: through a blower 230, and when the smoke emissions pass through a water tank filter 240, tar is further removed, and moisture is removed by a cloth filter 250. Thereafter, odor is eliminated by a zeolite filter 260 and an activated carbon filter 270, and then, from an intake duct 30, and the smoke emissions are fed to the thermal decomposition treatment chamber 110.

No new matter was added in the amendment.

### CROSS-REFERENCE TO RELATED APPLICATION

This application claims the benefit of Japanese Application No. 2015-022411 filed on Feb. 6, 2015 with the Japanese Patent Office, the disclosure of which is incorporated herein by reference.

### TECHNICAL FIELD

The present invention relates to a magnetic decomposition device and a magnetic decomposition method to thermally decompose waste, etc., by utilizing magnetically treated air.

### BACKGROUND ART

As a magnetic, decomposition device for waste, etc., for example, Patent Literature 1 describes an organic matter decomposition treatment device. In this device, an organic matter is charged into a bowl-shaped decomposition pot and ignited with an ignition rod, and magnetized air is introduced from an air introduction port of a magnetized air introduction pipe to magnetically burn the organic matter at a low temperature without extinguishing the fire, and accordingly, a combustion decomposition treatment is performed. Then, smoke containing tar and water vapor generated by said combustion decomposition treatment is made to rise, the tar and the water vapor are separated at a tar removal unit, and further, at a deodorization part, the smoke from which moisture and tar were removed is exposed in an atmosphere in which a deodorization liquid is sprayed and made odorless and discharged.

In an organic decomposition treatment device described in Patent Literature 2, smoke containing tar and moisture generated at the time of a combustion decomposition treatment of an organic matter charged into a decomposition chamber by magnetically burning the organic matter at a low temperature, is made to rise, most of the tar is removed at a tar removal unit, and the smoke is exposed in a watering atmosphere in a deodorizing unit and accordingly reduced and made odorless, and is then exhausted into the atmosphere.

### CITATION LIST

### Patent Literatures

Patent Literature 1: Japanese Unexamined Patent Application Publication No. 2010-75823

Patent Literature 2: Japanese Unexamined Patent Application Publication No. 2011-5457

### SUMMARY OF THE INVENTION

### Technical Problem

However, with the technologies described in Patent Literatures 1 and 2 listed above, although tar and odor are partially removed, removal is not sufficient, and eventually, smoke emissions containing tar, etc., are discharged. A filter that removes tar, etc., needs to be replaced or cleaned after elapse of a predetermined, period, and at the time of this replacement or cleaning, operation of the whole device must be stopped.

In view of the circumstances described above, an object of the present invention is to perform a magnetic decomposition treatment by effectively utilizing smoke emissions. Another object thereof is to replace or clean a filter without stopping operation of the whole device.

### Solution to the Problem

In order to attain the above-described objects, a magnetic decomposition device according to the present invention is a magnetic decomposition device that decomposes an object by performing magnetic decomposition utilizing air to which a magnetic field is applied, and includes: a decomposition treatment section that applies a decomposition treatment to the object; a filter section that is separated from the decomposition treatment section and filters smoke emissions generated by the decomposition treatment; a piping that feeds smoke emissions of the decomposition treatment section to the filter section and feeds smoke emissions filtered by the filter section to the decomposition treatment section; a magnet that is provided on the piping and applies a magnetic field to filtered smoke emissions; and a circulation means that circulates the smoke emissions between the decomposition treatment section and the filter section.

In addition, the decomposition treatment section starts a decomposition treatment by using a hot air gun.

In addition, the filter section includes, at an intake side of the circulation means, a means that lowers a temperature of smoke emissions.

In addition, the filter section has a filtering function to remove tar and eliminate odor.

In addition, on an inner wall of a thermal decomposition treatment chamber constituting the decomposition treatment section, a swashplate flue with an air intake part formed of an upward swashplate along an air flowing direction is formed.

In addition, the circulation means provided between the decomposition treatment section and the filter section is provided with a bypass means to suction a necessary amount that is a part of exhaust air from the decomposition treatment section into the decomposition treatment section.

In order to attain the above-described objects, a magnetic decomposition method according to the present invention is a magnetic decomposition method to decompose an object by performing magnetic decomposition utilizing air to which a magnetic field is applied, and includes the steps of: opening an upper shutter portion and charging an object into a hopper in a state where a lower shutter portion is closed; closing the upper shutter portion after the object is charged, into the hopper; opening the lower shutter portion after the upper shutter portion is closed, and charging the object into a thermal decomposition treatment chamber; and circulating air in at least the thermal decomposition treatment chamber, an exhaust duct, and an intake duct by driving a blower.

### Effects of the Invention

According to the present invention, smoke emissions generated in the decomposition treatment section are fed to the filter section, and after tar is removed and odor is eliminated by the filter section, returned to the decomposition treatment section while a magnetic field supplied by the magnet is applied thereto, and circulated, and therefore, a magnetic decomposition treatment can be performed by effectively utilizing smoke emissions. In addition, the filter section, is separated from the decomposition treatment section, so that without stoppage of operation of the whole device, the filter can be replaced and cleaned.

### BRIEF DESCRIPTION OF THE DRAWINGS

FIG. 1 is a plan view showing a magnetic decomposition device according to an embodiment of the present invention.

FIG. 2 is a cross-sectional plan view of a thermal decomposition treatment chamber of the magnetic decomposition device shown in FIG. 1.

FIG. 3 is a side view viewed from directions A1 and A2 in FIG. 1.

FIG. 4 is a side sectional view viewed from the directions A1 and A2 in FIG. 1.

FIG. 5 is a side sectional view of the thermal decomposition treatment chamber of the magnetic decomposition device shown in FIG. 1.

FIG. 6 is a developed side view viewed from directions A1 and A3 in FIG. 1.

### BEST MODE FOR CARRYING OUT THE INVENTION

Hereinafter, an embodiment of the present invention is described in detail with reference to the drawings.

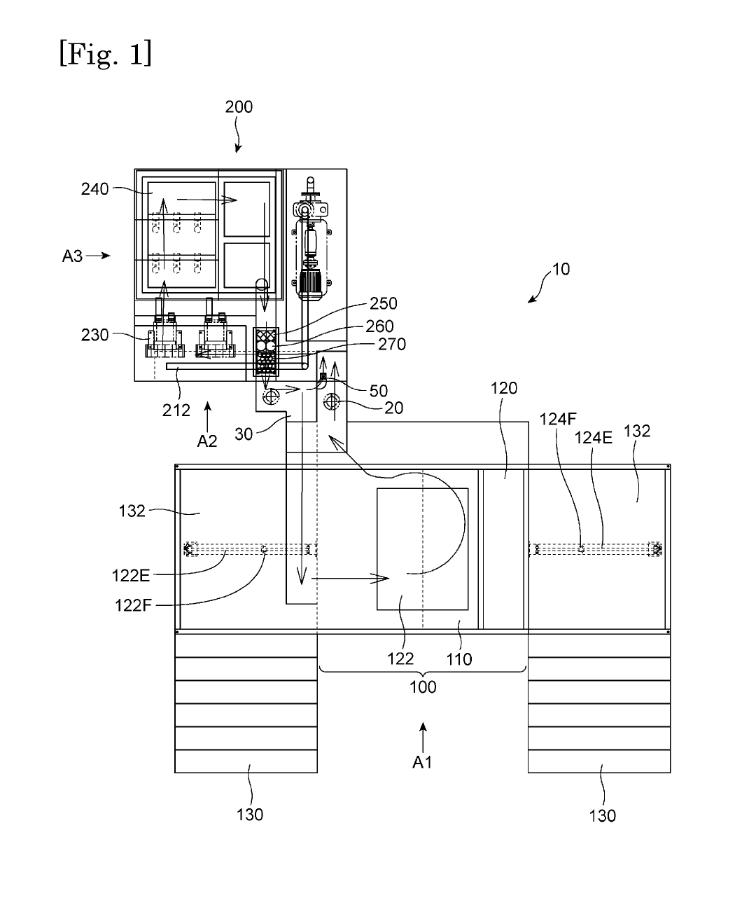

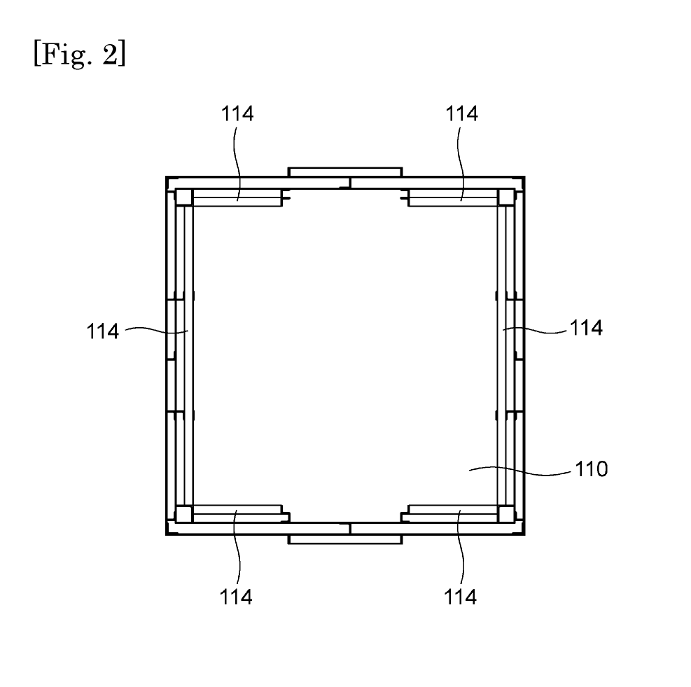

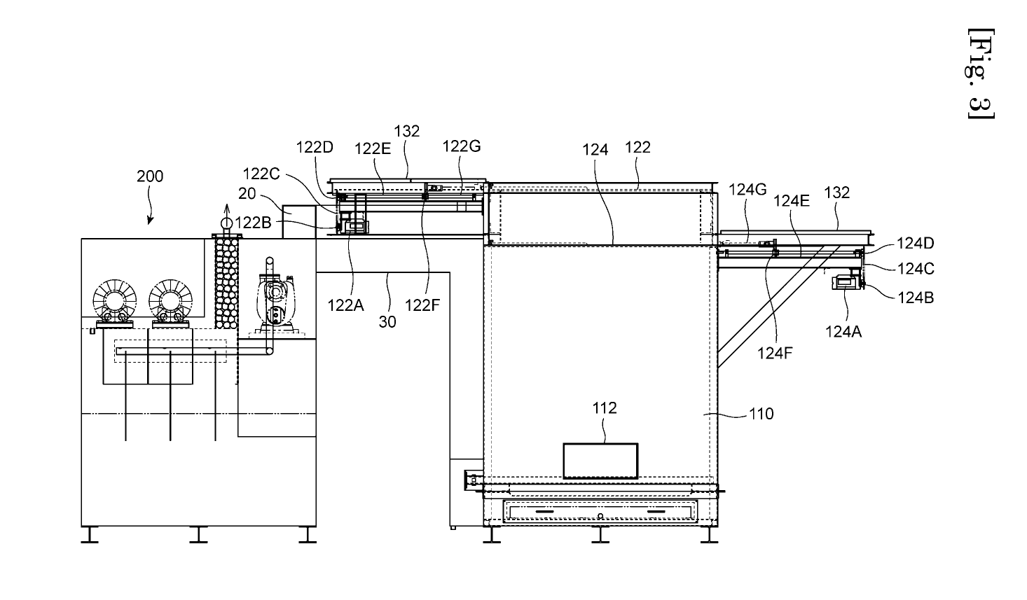

The present embodiment is shown in FIG. 1 to FIG. 6. In these drawings, the magnetic decomposition device 10 includes a decomposition treatment section 100 and a filter section 200. These sections are connected by an exhaust duct 20 and an intake duct 30, and smoke emissions circulate between the decomposition treatment section 100 and the filter section 200.

First, describing the decomposition treatment section 100, at an upper portion of a thermal decomposition treatment chamber 110 in which waste is decomposed, a hopper 120 into which waste is charged is provided. Those to be charged into the hopper 120 are various kinds of waste including household waste, waste paper, cardboard, waste plastic, wood waste, vegetable waste, rubber, arid vinyl, etc.

At an upper portion of the hopper 120, an upper shutter 122 is provided, and at a lower portion, a lower shutter 124 is provided. Stairs 130 for carrying waste from the ground to the hopper 120, and a charging step (floor) 132 are provided.

In order to charge waste, an operator climbs to the charging step 132 from the stairs 130, closes the lower shutter 124 and opens the upper shutter 122 of the hopper 120. Then, the operator charges waste into the hopper 120 and closes the upper shutter 122, and then opens the lower shutter 124. Accordingly, the waste inside the hopper 120 falls to the inside of the thermal decomposition treatment chamber 110. Next, the operator closes the lower shutter 124 again.

Thus, the upper shutter 122 is opened in the state where the lower shutter 124 is closed and waste is charged into the hopper 120, and the waste is made to fall to the inside of the thermal decomposition treatment chamber 110 by opening the lower shutter 124 in the state where the upper shutter 122 is closed, and accordingly, outside air (air not magnetically decomposed) flowing into the thermal decomposition treatment chamber 110 may be minimized. In addition, the hopper 120 is connected to the exhaust duct 20, and smoke emissions filling the inside of the hopper 120 can be exhausted to the filter section 200 by the above-described opening and closing operation, and therefore, the smoke emissions can be prevented from flowing to the outside.

The upper shutter 122 and the lower shutter 124 may be a motor-driven type or a hydraulic type as appropriate, and both of these shutters may open and close in conjunction with each other. In the example shown in the drawings, in response to an operation of a switch not shown, the upper shutter 122 is driven to open and close by driving and rotating a ball screw 122E via a geared motor 122A, a sprocket 122B, a roller chain 122C, and a sprocket 122D, and converting the rotational movement into linear movement of a shutter drive rod 122G by a drive screw 122F.

Similarly, in response to an operation of a switch not shown, the lower shutter 124 is driven to open and close by rotating and driving a ball screw 124E via a geared motor 124A, a sprocket 124B, a roller chain 124C, and a sprocket 124D, and converting the rotational movement into a linear movement of a shutter drive rod 124G by a drive screw 124F. The lower shutter 124 includes a pair of left and right shutter plates, and is driven by bilaterally symmetrical drive mechanisms.

Next, in a side surface of the thermal decomposition treatment chamber 110, an opening 112 is provided, and the opening 112 is closed during a decomposition treatment. This opening 112 is used to supply hot air flow to the inside of the treatment chamber at the start of a decomposition treatment, and remove residue after a decomposition treatment.

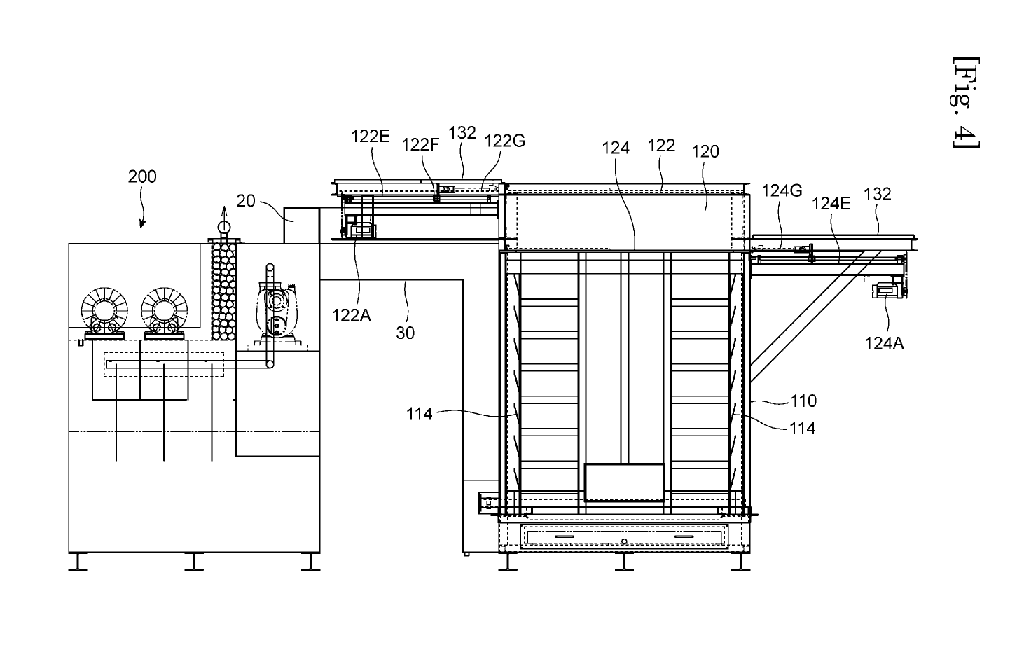

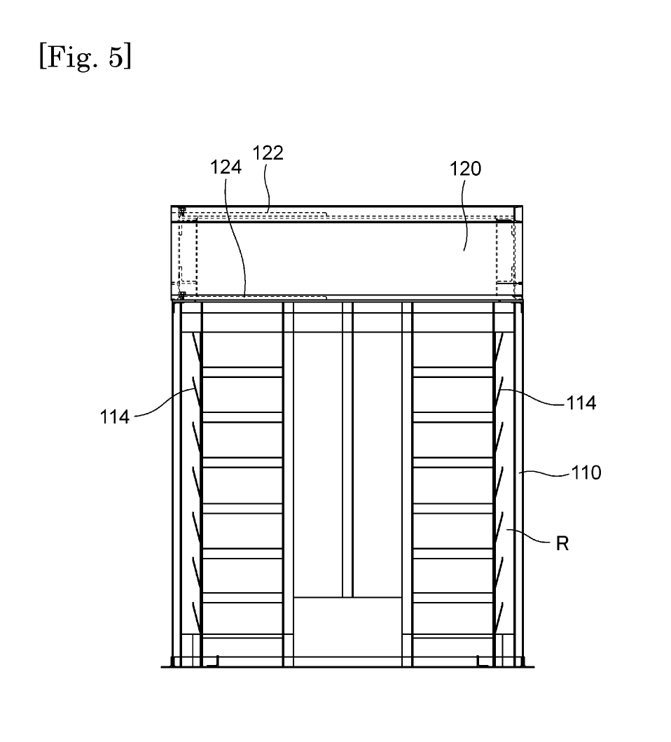

The inner wall of the thermal decomposition treatment chamber 110 mainly doubles as a flue, and is formed by using a material such as an iron plate that is a ferromagnetic body. In addition, on the inner wall of the thermal decomposition treatment chamber 110, as shown in FIG. 4 and FIG. 5, a swashplate flue 114 with an air intake part formed of an upward swashplate is formed, and wherever the position of waste charged into the thermal decomposition treatment chamber 110 is, smoke emissions generated by thermal decomposition of the waste are guided upward by the swashplate flue 114, and efficiently discharged into the exhaust duct 20.

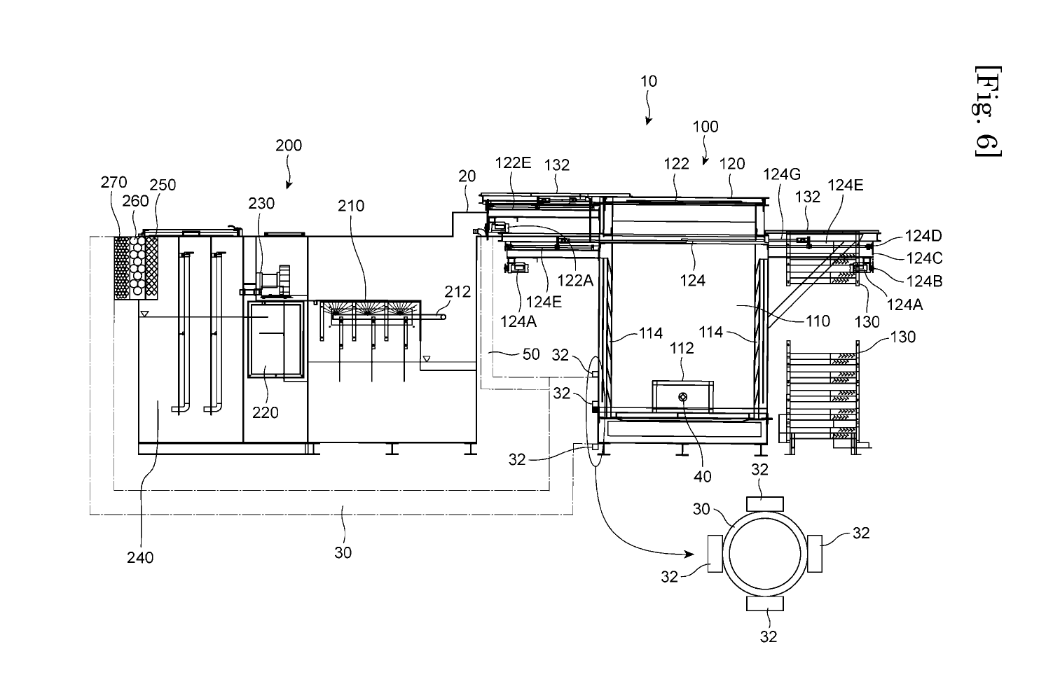

Next, the filter section 200 is described. The filter section 200 includes, as shown in FIG. 1 and FIG. 6, a plurality of filter units and a blower unit that circulates smoke emissions. Smoke emissions introduced from the above-described exhaust duct 20 pass through a shower 210, a swashplate filter 220, a blower 230, a water tank filter 240, a cloth filter 250, a zeolite filter 260, and an activated carbon filter 270 in this order inside the filter section 200, and reach the intake duct 30. By the blower 230, smoke emissions are circulated in the whole device.

Among these components, the shower 210 is arranged to spray smoke emissions with water from a watering pipe 212. The swashplate filter 220 is arranged to bring smoke emissions into contact with a plurality of plates disposed alternately diagonally. These shower 210 and swashplate filter 220 are provided to lower a temperature of smoke emissions, remove tar, and reduce a load on the blower 230 disposed at the rear stage of the shower and the swashplate.

Next, the water tank filter 24 0 is provided to remove mainly tar when smoke emissions descend into water in the form of bubbles. The cloth filter 250 is provided to remove moisture from smoke emissions by cloth. The zeolite filter 260 is mainly to exhibit a smoke elimination effect, and remove colors. The activated carbon filter 270 is provided to eliminate mainly odor of smoke emissions. Smoke emissions from which tar and odor were removed by these filters can be released into the atmosphere without problems, however, in the present embodiment, the smoke emissions pass through an iron pipe through the intake duct 30 and return to the thermal decomposition treatment chamber 110. At this time, the smoke emissions are magnetically decomposed by a powerful magnetic field generated by magnets 32 provided on the intake duct 30. The magnets 32 are attached to the outside of the iron pipe projecting to the outside of the thermal decomposition treatment, chamber 110.

As the magnets 32, permanent magnets such as Nd—Fe—B magnets (neodymium magnets) or Sm—Co magnets (samarium-cobalt magnets) are preferably used by way of example. By using permanent magnets, a powerful magnetic field can be obtained without requiring other energy sources. When the powerful magnetic field generated by the magnets 32 acts on air, oxygen and nitrogen in the air are separated, however, a magnetic susceptibility of oxygen is 1000 times as high as that of nitrogen, and this acts on waste and the waste is thermally decomposed. In order to create a powerful magnetic field, as the magnets 32, permanent magnets with a magnetic flux density of, for example, approximately 700 mT (7000 gauss) or more are used.

Next, in the present embodiment, the treatment is started by using a hot air gun 40. Accordingly, without use of fire, a decomposition treatment is started and continued. In detail, the opening 112 of the thermal decomposition treatment chamber 110 is opened, and by blowing hot air from this opening into the chamber by the hot air gun 40 and driving the blower 230, smoke emissions are magnetically decomposed and circulated, and accordingly, the decomposition treatment is continuously performed.

Next, the whole operation of the present embodiment is described. First, in a state where the lower shutter 124 is closed, the upper shutter 122 is opened and waste is charged into the hopper 120, and in a state where the upper shutter 122 is closed, the lower shutter 124 is opened to make the waste fall into the thermal decomposition treatment chamber 110. On the other hand, by driving the blower 230, air is circulated between the decomposition treatment section 100 and the filter section 200 through the exhaust duct 20 and the intake duct 30. Accordingly, the air is magnetically separated by a magnetic field of the magnets 32 provided on the intake duct 30, and the magnetically treated air is fed into the thermal decomposition treatment chamber 110.

Concerning intake air and exhaust air of the decomposed treatment section 110, smoke emissions expanded by thermal decomposition are exhausted, however, intake air is not forcibly fed but only a necessary amount of intake air is fed by a bypass piping 50. Thus, by providing the bypass piping 50, only a necessary amount of the gas in the intake duct 30 can be introduced into the thermal decomposition treatment chamber 110, and an unnecessary amount of the gas can be diverted to the exhaust duct 20 through the bypass piping 50, so that a pressure inside the thermal decomposition treatment chamber that easily turns into a pressurized state is reduced and a heat source is made stable in an excellent state, and accordingly, an advantage of continuously maintaining an optimum treatment environment is obtained.

In this state, the opening 112 of the thermal decomposition treatment chamber 110 is opened and hot air at, for example, approximately 500° C. is blown into the chamber by the hot air gun 40. Then, the waste is thermally decomposed inside the thermal decomposition treatment chamber 110. Air blowing by the hot air gun 40 can be performed for about 3 minutes, and thereafter, thermal decomposition continues. A temperature inside the thermal decomposition treatment chamber 110 is approximately 400 to 500° C., and combustion does not occur inside.

Smoke emissions generated by the thermal decomposition are fed to the filter section 200 through the exhaust duct 20.

When the smoke emissions pass through the shower 210 and the swashplate filter 220 of the filter section 200, the temperature of the smoke emissions is lowered and tar is removed therefrom, and then the smoke emissions pass through the blower 230. When the smoke emissions pass through the water tank filter 240, tar is further removed, and next, moisture is removed by the cloth filter 250. Thereafter, odor of the smoke emissions is eliminated by the zeolite filter 260 and the activated carbon filter 270, and then the smoke emissions are fed from the intake duct 30 to the thermal decomposition treatment chamber 110. At this time, the smoke emissions are subjected to a magnetic field by the magnets 32, and utilized for a thermal decomposition treatment.

Here, water tanks for the shower 210 and the water tank filter 240 are separated, and a surfactant is mixed in a range of 1% to 5% into the water tank for the shower 210. In addition, sodium hydroxide is mixed in a range of 1% to 5% into the water tank for the water tank filter 240, and made to adsorb carbon dioxide contained in the smoke emissions. Accordingly, the gas that passes through the intake duct 30 and returns to the thermal decomposition treatment chamber 110 can be made cleaner.

As described above, the present embodiment brings about the following effects.

a. Smoke emissions generated in the decomposition treatment section 100 are fed into the filter section 200 from the exhaust duct 20, and subjected to tar removal and odor elimination by the filter section 200, and then, returned to the decomposition treatment section 100 while being subjected to a magnetic field by the magnets 32 provided on the intake duct 30, and circulated, and therefore, a magnetic decomposition treatment can be performed by effectively utilizing the smoke emissions.

b. The filter section 200 is separated from the decomposition treatment section 100, so that by preparing a plurality of filter sections 200 and switching connections of exhaust ducts 20 and intake ducts 30, while maintenance of a used filter section 200 is performed, the decomposition treatment can be performed by using a new filter section 200, and therefore, without stoppage of operation of the entire device, the decomposition treatment can be continued.

c. Thermal decomposition is started by using a hot air gun, so that waste can be safely treated without use of fire.

d. A filter that lowers a temperature of smoke emissions and a filter that removes tar are provided at the front stage of the blower that circulates smoke emissions, so that the load on the blower is reduced.

The present invention is not limited to the above-described embodiment, and can be variously changed without departing from the spirit of the present invention. For example, the present invention also includes the following.
(1) Shapes and dimensions shown in the embodiment described above are just examples, and the design can be changed so that the same operation and effects are exhibited.
(2) In the embodiment described above, the shower 210 and the swashplate filter 220 are provided at the front stage of (on the intake side of) the blower 230, and the water tank filter 240, the cloth filter 250, the zeolite filter 260, and the activated carbon filter 270 are provided at the rear stage of (on the exhaust side of) the blower 230, however, which position which filter is provided at can be properly set as necessary.

### INDUSTRIAL APPLICABILITY

According to the present invention, the filter section is separated from the decomposition treatment section, and between these, a magnetic field is applied and smoke emissions are circulated, so that the present invention is preferable for decomposition of various types of waste.

### REFERENCE SIGNS LIST

10 Magnetic decomposition device
20 Exhaust duct
30 Intake duct
32 Magnet
40 Hot air gun
50 Bypass piping
100 Decomposition treatment section
110 Thermal decomposition treatment chamber
112 Opening
114 Swashplate flue
120 Hopper
122 Upper shutter
122A Geared motor (gear motor)
122B Sprocket:
122C Roller chain
122D Sprocket
122E Ball screw
122F Drive screw
122G Shutter drive rod
124 Lower shutter
124A Geared motor (gear motor)
124B Sprocket
124C Roller chain
124D Sprocket
124E Ball screw
124F Drive screw
124G Shutter drive rod
130 Stairs
132 Charging step
200 Filter section
210 Shower
212 Watering pipe
220 Swashplate filter
230 Blower
240 Water tank filter
250 Cloth filter
260 Zeolite filter
270 Activated carbon filter

1. A magnetic decomposition device that decomposes waste by performing magnetic decomposition utilizing air to which a magnetic field is appiled, comprising:
a decomposition treatment section that applies a decomposition treatment to the waste;
a blower that circulates smoke emissions discharged from the decomposition treatment section;
a filter section that includes a shower having a watering pipe and disposed at a front stage of the blower in a flowing direction of the smoke emissions, and a water tank filter disposed at a rear stage of the blower in the flowing direction of the smoke emissions;
an exhaust duct that feeds smoke emissions of the decomposition treatment section to the filter section;
an intake duct that feeds smoke emissions filtered by the filter section to the decomposition treatment section; and
a magnet, that is provided on the intake duct and applies a magnetic field to filtered smoke emissions.

2. The magnetic decomposition device according to claim 1, wherein the decomposition treatment section starts the decomposition treatment by using a hot air gun.

3. The magnetic decomposition device according to claim 1, wherein the filter section includes a swashplate filter formed of a plurality of plates disposed alternately diagonally at the front stage of the blower and at the rear stage of the shower.

4. The magnetic decomposition device according to claim 1, wherein on an inner wall of a thermal decomposition treatment chamber of the decomposition treatment section, a swashplate flue with an air intake part formed of an upward swashplate along an air flowing direction is formed.

5. The magnetic decomposition device according to claim 1, wherein a bypass piping for a part of the smoke emissions flowing in the intake duct to the exhaust duct to bypass the decomposition treatment section is provided.

6. A magnetic decomposition method to decompose waste by performing magnetic decomposition utilizing air to which a magnetic field is applied, comprising the steps of:
charging a decomposition treatment section with the waste and thermally decomposing the waste;
circulating smoke emissions discharged during the thermal decomposing step with a blower;
feeding smoke emissions discharged from the decomposition treatment section to a shower that is disposed at a front stage of the blower through an exhaust duct, the shower having watering pipe;
feeding smoke emissions discharged from the shower to a water tank filter disposed at a rear stage of the blower; and
feeding smoke emissions discharged from the water tank filter to the decomposition treatment section through an intake duct.

7. (canceled)
